(19) United States
(12) Patent Application Publication
(10) Pub. No.: US 2019/0350883 A1
(43) Pub. Date: Nov. 21, 2019

(54) Pharmaceutical Composition of Acetaminophen/Ibuprofen

(71) Applicant: Luan Chau, Greenville, SC (US)

(72) Inventor: Luan Chau, Greenville, SC (US)

(21) Appl. No.: 16/416,369

(22) Filed: May 20, 2019

Related U.S. Application Data
(60) Provisional application No. 62/674,001, filed on May 20, 2018.

(51) Int. Cl. A61K 31/167; A61K 31/192; A61K 9/20; A61K 9/28

(52) U.S. Cl. CPC A61K 31/167; A61K 31/192; A61K 9/28; A61K 9/2054; A61K 9/2059; A61K 9/2027

(57) ABSTRACT

A solid oral dosage form comprising therapeutically effective amounts of acetaminophen and a non-steroidal anti-inflammatory drug (NSAID) as active ingredients. The dosage form may contain binder material to cohesively bind the ingredients. The dosage form may further comprise a disintegrant to ensure effective disintegration in the gastrointestinal tract. For example, the solid oral dosage form may be a single tablet that contains about 1,000 mg of acetaminophen and about 400 mg of ibuprofen. In another example, the solid oral dosage form may be a single tablet that contains about 650 mg of acetaminophen and about 400 mg of ibuprofen. This invention may be particularly suitable in the management of dental pain.

### TECHNICAL FIELD

This invention relates to solid pharmaceutical dosage formulations of analgesic medications for the treatment of pain.

### BACKGROUND

The dual goals of pain management are safety and efficacy. Opioid medications have long been widely used in the management of acute pain, especially in the post-surgical setting. Although abuse of opioid medications has always been a concern, it has recently reached crisis levels because of over-prescription and widespread availability. In 2016, the Centers for Disease Control & Prevention recommended limiting the dose and duration of opioid-containing medications. Indeed, there is increased scrutiny of clinicians' choice of analgesic medications for managing acute pain.

These circumstances have led clinical practitioners to seek alternatives for effective management of pain in their patients. There are numerous non-opioid medications that are available for this purpose, most particularly, acetaminophen and nonsteroidal anti-inflammatory drugs (NSAIDs). However, these are conventionally perceived to be less potent and less effective than opioid medications.

However, a more recent analysis of the relevant clinical literature has proposed that the combination of 400 mg of ibuprofen plus 1,000 mg of acetaminophen is actually superior to opioid-containing medications for the treatment of acute dental pain. See Paul Moore et al, “Benefits and harms associated with analgesic medications used in the management of acute dental pain: An overview of systematic reviews” J Am Dent Assoc. (2018 April), 49(4):256-265. As such, it would be beneficial to provide a more convenient formulation that allows patients to take this as a single dose.

### SUMMARY

This invention provides a pharmaceutical composition as a solid oral dosage form. As used herein, “solid oral dosage form” means any orally ingestible form for oral administration, including tablets, capsules, powders, sachets and the like. As used herein, “tablet” means a compressed or molded solid dosage form of any shape or size, and includes caplets.

In one aspect, the invention is a pharmaceutical tablet (single unit) comprising a therapeutically effective amount of ibuprofen and a therapeutically effective amount of acetaminophen. The tablet further comprises excipient material that consists of one or more excipients. The excipient(s) includes binder material. The amount of excipient material or binder material in the tablet may be limited to ensure a tablet size suitable for oral ingestion. The tablet of the invention could be made using any suitable manufacturing techniques such as direct compression, wet granulation, or dry granulation.

In another aspect, the invention is a pharmaceutical capsule (single unit) having contents that comprise of a therapeutically effective amount of ibuprofen, a therapeutically effective amount of acetaminophen, and less than 5 wt % of excipient material (if any). The capsule size is sufficiently large to encapsulate the therapeutically effective amount of the active ingredients and any excipients. In some embodiments, the capsule is standard size 00 or 000.

In another aspect, the invention is a method of treating pain in a patient comprising ingesting or administering a solid oral dosage form of the invention. The patient may receive intermittent dosing, for example, with a dosing frequency of every 4-6 hours.

### BRIEF DESCRIPTION OF THE DRAWINGS

FIGS. 1A and 1B show the dimensions of an example tablet of the invention. FIG. 1A shows a top view. FIG. 1B shows a side view.

FIGS. 2A and 2B shown different standard capsule sizes. FIG. 2A depicts a standard size 00 capsule. FIG. 2B depicts a standard size 000 capsule.

### DETAILED DESCRIPTION

In the solid oral dosage form, the active pharmaceutical ingredients consist of acetaminophen (also known as paracetamol) and a nonsteroidal anti-inflammatory drug (NSAID). A therapeutically effective amount of both active ingredients are loaded into a single unit of the solid oral dosage form. As such, the solid oral dosage form may have a high load of the active ingredients. In some embodiments, the dosage form contains at least 75 wt % of the two active ingredients; in some cases, at least 80 wt %; in some cases, at least 83 wt %; in some cases, at least 86 wt %; in some cases, at least 89 wt %; and in some cases, at least 92 wt %. In the context of a tablet, the term “wt %” means weight percent relative to the total weight of the tablet. In the context of a capsule, the term “wt %” means weight percent relative to the total weight of the capsule contents.

### 1. ACETAMINOPHEN

The solid oral dosage form comprises a therapeutically effective amount of acetaminophen. As used herein, the term “acetaminophen” also includes any pharmaceutically acceptable isomer, ester, polymorph, or salt thereof. Any suitable therapeutically effective amount of the acetaminophen may be used. In some embodiments, a single unit of the solid oral dosage form contains 900-1,100 mg of acetaminophen; and in some cases, about 1,000 mg (i.e. ±5%). In some embodiments, a single unit of the solid oral dosage form contains 580-715 mg of acetaminophen; and in some cases, about 650 mg (i.e. ±5%).

### 2. NSAID

The solid oral dosage form comprises a therapeutically effective amount of a non-steroidal anti-inflammatory drug (NSAID). Examples of suitable NSAIDs include propionic acid derivatives such as ibuprofen, naproxen, flurbiprofen, fenbufen, fenoprofen, indoprofen, ketoprofen, fluprofen, pirprofen, carprofen, oxaprozin, pranoprofen, suprofen, and the like; or acetic acid derivatives such as indomethacin, diclofenac, sulindac, tolmetin, and the like; or fenamic acid derivatives such as mefanamic acid, meclofenamic acid, flufenamic acid, and the like; or biphenylcarboxylic acid derivatives such as diflunisal, flufenisal, and the like; or oxicams such as piroxicam, sudoxicam, isoxicam, meloxicam, and the like.

In some embodiments, the NSAID is ibuprofen. As used herein, the term “ibuprofen” or any other named NSAID active ingredient includes any pharmaceutically acceptable isomer, ester, polymorph, or salt thereof. Any suitable therapeutically effective amount of the ibuprofen may be used. In some embodiments, a single unit of the solid oral dosage form contains 360-440 mg of ibuprofen; and in some cases, about 400 mg (i.e. ±5%).

In some embodiments, a single unit of the solid oral dosage form contains acetaminophen and ibuprofen in a ratio of 2:1: to 3:1 by weight (acetaminophen:ibuprofen). In some embodiments, a single unit of the solid oral dosage form contains acetaminophen and ibuprofen in a ratio of 1.3:1 to 2:1 by weight (acetaminophen:ibuprofen).

### 3. BINDER

The solid oral dosage form may contain one or more binders. Binders have adhesive property and serve to promote cohesiveness in the solid dosage form. There are many different types of binder ingredients that could be used. Examples of binders that could be used include saccharides and their derivatives, such as disaccharides (e.g. sucrose, lactose); polysaccharides and their derivatives, such as starches, cellulose or modified cellulose such as microcrystalline cellulose and cellulose ethers such as hydroxypropyl cellulose (HPC); sugar alcohols such as xylitol, sorbitol or mannitol; proteins such as gelatin; and synthetic polymers such as polyvinylpyrrolidone (PVP), polyethylene glycol (PEG), etc.

Particularly useful in this invention are binder materials having high compactibility. One example of a highly compactible and strong binder is microcrystalline cellulose (MCC), which is particularly useful in direct compression manufacturing of tablets. In some embodiments, the microcrystalline cellulose is microcrystalline cellulose type 101. In some embodiments, the microcrystalline cellulose is microcrystalline cellulose type 102. In some embodiments, the microcrystalline cellulose binder is a mixture of microcrystalline cellulose 101 and microcrystalline cellulose 102 in a ratio of 1:1 or higher (MCC 101:MCC 102), and in some cases, in a ratio of 1:1.2 or higher. Other highly compactible binders that could be particularly suitable are polyvinylpyrrolidone (also known as PVP or povidone) and pregelatinized starch.

With the high drug load already increasing the size of the oral dosage form, the amount of binder material should be limited to avoid excessive size of the oral dosage form (to allow for oral ingestion). As such, in some embodiments, the total binder material (consisting of one or more binder ingredients) may be less than 21 wt %; in some cases, less than 19 wt %; in some cases, less than 17 wt %; in some cases, less than 15 wt %; in some cases, less than 13%; in some cases, less than 11 wt %; in some cases, less than 9 wt %; in some cases, less than 7 wt %; in some cases, less than 5 wt %. In the context of a tablet, the term “wt %” means weight percent relative to the total weight of the tablet. In the context of a capsule, the term “wt %” means weight percent relative to the total weight of the capsule contents.

Because the binder is essential to cohesively binding the ingredients together into a tablet, the binder ingredient(s) may constitute a relatively larger fraction of the excipient material. In some embodiments, of the total amount of excipient material (consisting of one or more excipient ingredients) in the dosage form, the binder material (consisting of one or more binder ingredients) constitutes at least 70 wt % relative to the total amount of excipients (weight percent relative to the total weight of all the excipient ingredients of a tablet or of the contents of a capsule); in some cases, at least 73 wt %; in some cases, at least 76 wt %; in some cases, at least 79 wt %; in some cases, at least 82 wt %; in some cases, at least 85 wt %; in some cases, at least 88 wt %; in some cases, at least 91 wt %; and in some cases, at least 94 wt %.

### 4. DISINTEGRANTS

The solid oral dosage form may contain one or more disintegrants. Disintegrants are often included in pharmaceutical formulations to ensure an acceptable disintegration rate in the gastrointestinal tract. As used herein, the term “disintegrant” means a material in the formulation that acts to cause the solid matrix to break up when the solid dosage form comes into contact with aqueous media. Typical disintegrants include starch derivatives, salts of carboxymethyl cellulose, and crosslinked polymers of povidone. In some embodiments, the disintegrant may be a superdisintegrant such as sodium starch glycolate, croscarmellose sodium, or crospovidone. (Crospovidone is cross-linked polyvinylpyrrolidone or cross-linked povidone.)

With the high drug load already increasing the size of the oral dosage form, the amount of disintegrant material should be limited to avoid excessive size of the oral dosage form (to allow for oral ingestion). As such, in some embodiments, the total amount of disintegrant material (consisting of one or more disintegrant ingredients) in the dosage form is less than 9 wt %; in some cases, less than 7 wt %; in some cases, less than 5 wt %; in some cases, less than 4 wt %; in some cases, less than 3 wt %; in some cases, less than 2 wt %. In the context of a tablet, the term “wt %” means weight percent relative to the total weight of the tablet. In the context of a capsule, the term “wt %” means weight percent relative to the total weight of the capsule contents.

### 5. LUBRICANTS

The solid oral dosage form may contain one or more lubricants. Lubricants prevent the pharmaceutical ingredients from clumping together and from sticking to the tablet punches or capsule filling machine. As such, lubricant material can be added in small amounts to improve processing characteristics. Examples of commonly used lubricants include talc, silica, and fats (such as vegetable stearin, magnesium stearate, calcium stearate, stearic acid).

### 6. OTHER EXCIPIENTS

As used herein, the term “excipient” means materials other than the two active pharmaceutical ingredients, i.e. the acetaminophen and NSAID. Examples of other excipient materials include glidants (e.g. colloidal silicon dioxide), flavorants (e.g. menthol, peppermint, mint flavors, fruit flavors, chocolate, vanilla, bubblegum flavors, coffee flavors, etc.), preservatives (e.g. citric acid, sodium citrate, vitamin A, vitamin E, vitamin C), and diluents. Excipient ingredients may also be used for making a film coating.

### 7. EXCIPIENT LOAD

With the high drug load already increasing the size of the oral dosage form, the amount of excipient(s) should be limited to avoid excessive size of the oral dosage form (to allow for oral ingestion). As such, in some embodiments, the total amount of excipient material (consisting of one or more excipient ingredients, including any used for a tablet coating or film) in the solid oral dosage form is less than 18 wt %; in some cases, less than 15 wt %; in some cases, less than 12 wt %; in some cases, less than 9 wt %; in some cases, less than 7 wt %; and in some cases, less than 5 wt %. In the context of a tablet, the term “wt %” means weight percent relative to the total weight of the tablet. In the context of a capsule, the term “wt %” means weight percent relative to the total weight of the capsule contents.

### 8. SHAPE & DIMENSIONS

In embodiments in which the solid dosage form is a tablet, the tablet may have any suitable shape and size for oral ingestion. Examples of tablet shapes include round, oval, modified oval, elliptical, cylindrical, etc. In some embodiments, the tablet has an elongate shape (e.g. oval, elliptical, cylindrical, etc.).

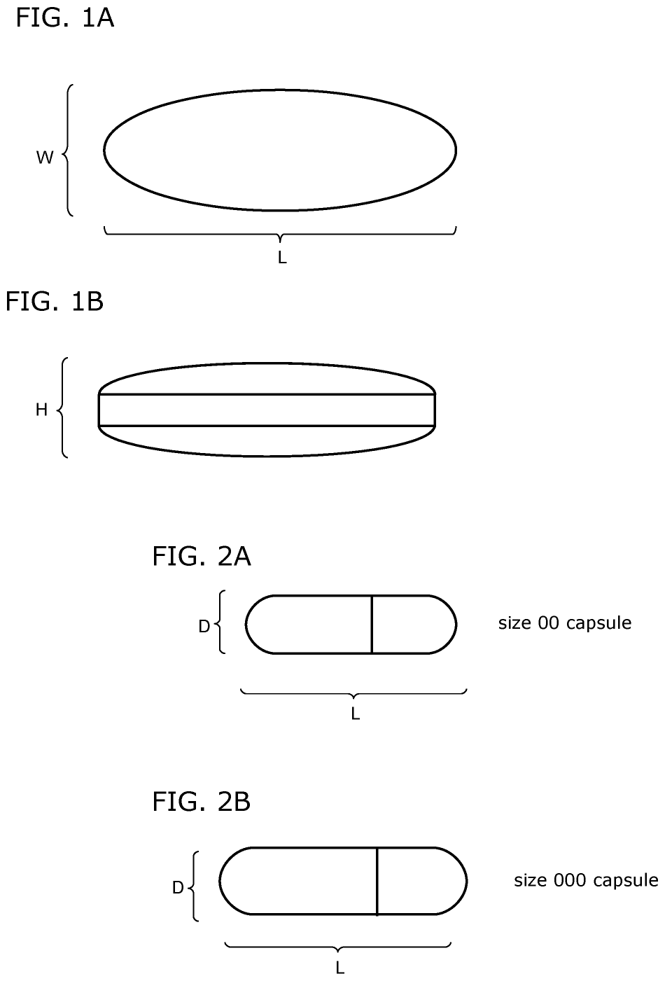

Herein, the largest dimension of a tablet form is referred to as the “length,” the second largest dimension is referred to as the “width,” and the smallest dimension is referred to as the “height.” FIGS. 1A and 1B demonstrates measurement of the dimensions of an example tablet. FIG. 1A shows a top view of the tablet with “L” indicating the length and “W” indicating the width. FIG. 1B shows a side view with “H” indicating the height. If the tablet shape is symmetrical along one or more axes, then one or more of such dimensional parameters may be superfluous. For example, if the tablet has a cylindrical shape, then the width and height would be the same.

In some embodiments, the tablet has an elongate shape and the length is 23 mm or less; in some cases, 21 mm or less; and in some cases, 19 mm or less. In some embodiments, the tablet has a width of 14 mm or less; in some cases, 12 mm or less; and in some cases, 10 mm or less.

In some embodiments, the solid dosage form is an elongate-shaped capsule, which may be of any suitable capsule size. The dimensions of different standard capsule sizes are shown in FIGS. 2A and 2B. In FIG. 2A, a size 00 capsule is depicted. The size 00 capsule has a volume of 0.95 ml, a locked length (L) of 23.3 mm, and an external diameter (D) of 8.53 mm. FIG. 2B depicts a standard size 000 capsule. The size 000 capsule has a volume of 1.37 ml, a locked length (L) of 26.14 mm, and an external diameter (D) of 9.91 mm.

### 9. TREATMENT METHOD & DOSING

The pharmaceutical composition of this invention could be used to treat pain, such as post-surgical pain. One particular example is acute dental pain. The solid oral dosage form may be dosed in any suitable manner to achieve a therapeutic effect. A used herein, the term “a dose” means a single unit of the solid oral dosage form (e.g. a single tablet or a single capsule); likewise, “dosing” means the ingestion or administration of a single dose.

The patient may receive intermittent dosing. In some embodiments, the dosing frequency is every 3.5-6.5 hours; and in some cases, every about 4-6 hours. There may be a maximum adult dose that should not be exceeded. In some embodiments, the maximum adult daily amount is 3 or 4 single units of the oral dosage form daily or in a 24 hour period.

### 10. EXAMPLES

This invention will be further illustrated by the following specific hypothetical examples. The order of steps, the amount of the ingredients, inclusion of other optional ingredients, and other process variables can be determined, modified, or adjusted through routine empirical exercises.

In Example 1, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine pregelatinized starch (binder), microcrystalline cellulose (binder), and crospovidone (disintegrant) and mix together. Pass the excipient mixture through a sieve to sift out excessively large clumps. Add the excipient blend to the acetaminophen/ibuprofen blend and mix together to make a dry powder blend. Test the powder blend for adequate flowability. Compress the dry powder blend on a rotary tablet press to generate tablets having the composition in the table below.

Example 1: Direct Compression, total tablet weight = 1,523 mg

| | | | | |
|---|---|---|---|---|
| Acetaminophen (active ingredient #1) | 1,000 | mg | 66 | wt % |
| Ibuprofen (active ingredient #2) | 400 | mg | 26 | wt % |
| Microcrystalline cellulose | 46 | mg | 3 | wt % |
| Pregelatinized starch | 46 | mg | 3 | wt % |
| Crospovidone (crosslinked PVP) | 31 | mg | 2 | wt % |

In Example 2, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine polyvinylpyrrolidone (binder), croscarmellose sodium (disintegrant), and magnesium stearate (lubricant) and mix together. Pass the excipient mixture through a sieve to sift out excessively large clumps. Add the excipient blend to the acetaminophen/ibuprofen blend and mix together to make a dry powder blend. Test the powder blend for adequate flowability. Compress the dry powder blend on a rotary tablet press to generate tablets having the composition in the table below.

Example 2: Direct Compression, total tablet weight = 1,505 mg

| | | | | |
|---|---|---|---|---|
| Acetaminophen (active ingredient #1) | 1,000 | mg | 66 | wt % |
| Ibuprofen (active ingredient #2) | 400 | mg | 27 | wt % |
| Polyvinylpyrrolidone (non-crosslinked PVP, | 82.5 | mg | 5.5 | wt % |
| povidone) | | | | |
| Croscarmellose sodium | 15 | mg | 1 | wt % |
| Magnesium stearate | 7.5 | mg | 0.5 | wt % |

In Example 3, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine polyvinylpyrrolidone (binder) and sodium starch glycolate (disintegrant) and mix together. Pass the excipient mixture through a sieve to sift out excessively large clumps. Add the excipient blend to the acetaminophen/ibuprofen blend and mix together. Granulate the mixture by spraying with ethanol 95% and mixing. Pass the granulate through a sieve.

Dry the granulate on a fluid bed dryer and pass through a sieve. Add magnesium stearate (lubricant) to the dried granulate and place into a tumbler mixer for mixing. Test the dry granulate mixture for adequate flowability. Compress the granulate blend on a rotary tablet press to generate tablets having the composition in the table below.

Example 3: Wet Granulation, total tablet weight = 1,591 mg

| | | | | |
|---|---|---|---|---|
| Acetaminophen (active ingredient #1) | 1,000 | mg | 63 | wt % |
| Ibuprofen (active ingredient #2) | 400 | mg | 25 | wt % |
| Polyvinylpyrrolidone (non-crosslinked PVP, | 111 | mg | 7 | wt % |
| povidone) | | | | |
| Sodium starch glycolate | 64 | mg | 4 | wt % |
| Magnesium stearate | 16 | mg | 1 | wt % |

In Example 4, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine pregelatinized starch (binder) and magnesium stearate (lubricant) and mix together. Pass the excipient mixture through a sieve to sift out excessively large clumps. Add the excipient blend to the acetaminophen/ibuprofen blend and mix together. Granulate the mixture by spraying with purified water and mixing. Pass the granulate through a sieve.

Dry the granulate on a fluid bed dryer and pass through a sieve. Add magnesium stearate (lubricant) to the dried granulate and place into a tumbler mixer for mixing. Test the dry granulate mixture for adequate flowability. Compress the granulate blend on a rotary tablet press to generate tablets having the composition in the table below.

Example 4: Wet Granulation, total tablet weight = 1,543 mg

| | | | | |
|---|---|---|---|---|
| Acetaminophen (active ingredient #1) | 1,000 | mg | 65 | wt % |
| Ibuprofen (active ingredient #2) | 400 | mg | 26 | wt % |
| Pregelatinized starch | 139 | mg | 9 | wt % |
| Magnesium stearate | 4 | mg | 0.25 | wt % |

In Example 5, amounts of the various ingredients for the batch are scaled in proportion to achieve the per-capsule amounts shown in the table below. Combine ibuprofen and acetaminophen and mix together. Add magnesium stearate (lubricant) to the powder blend and continue mixing together until thoroughly mixed. Fill capsules of size “000” with the dry powder blend.

Example 5: Capsule, total content weight = 1,414 mg

| | | | | |
|---|---|---|---|---|
| Acetaminophen (active ingredient #1) | 1,000 | mg | 71 | wt % |
| Ibuprofen (active ingredient #2) | 400 | mg | 28 | wt % |
| Magnesium stearate (lubricant) | 14 | mg | 1 | wt % |

In Example 6, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine pregelatinized starch (binder), microcrystalline cellulose (binder), and crospovidone (disintegrant) and mix together. Pass the excipient mixture through a sieve to sift out excessively large clumps. Add the excipient blend to the acetaminophen/ibuprofen blend and mix together to make a dry powder blend. Test the powder blend for adequate flowability. Compress the dry powder blend on a rotary tablet press to generate tablets having the composition in the table below.

Example 6: Direct Compression, total tablet weight = 1,200 mg

| | | | | |
|---|---|---|---|---|
| Acetaminophen (active ingredient #1) | 650 | mg | 54.2 | wt % |
| Ibuprofen (active ingredient #2) | 400 | mg | 33.3 | wt % |
| Microcrystalline cellulose | 55 | mg | 4.6 | wt % |
| Pregelatinized starch | 55 | mg | 4.6 | wt % |
| Crospovidone (crosslinked PVP) | 40 | mg | 3.3 | wt % |

In Example 7, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine polyvinylpyrrolidone (binder), croscarmellose sodium (disintegrant), and magnesium stearate (lubricant) and mix together. Pass the excipient mixture through a sieve to sift out excessively large clumps. Add the excipient blend to the acetaminophen/ibuprofen blend and mix together to make a dry powder blend. Test the powder blend for adequate flowability. Compress the dry powder blend on a rotary tablet press to generate tablets having the composition in the table below.

Example 7: Direct Compression, total tablet weight = 1,192 mg

| | | | | |
|---|---|---|---|---|
| Acetaminophen (active ingredient #1) | 650 | mg | 54.5 | wt % |
| Ibuprofen (active ingredient #2) | 400 | mg | 33.6 | wt % |
| Polyvinylpyrrolidone (non-crosslinked PVP, | 100 | mg | 8.4 | wt % |
| povidone) | | | | |
| Croscarmellose sodium | 35 | mg | 2.9 | wt % |
| Magnesium stearate | 7 | mg | 0.6 | wt % |

In Example 8, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine polyvinylpyrrolidone (binder) and sodium starch glycolate (disintegrant) and mix together. Pass the excipient mixture through a sieve to sift out excessively large clumps. Add the excipient blend to the acetaminophen/ibuprofen blend and mix together. Granulate the mixture by spraying with ethanol 95% and mixing. Pass the granulate through a sieve.

Dry the granulate on a fluid bed dryer and pass through a sieve. Add magnesium stearate (lubricant) to the dried granulate and place into a tumbler mixer for mixing. Test the dry granulate mixture for adequate flowability. Compress the granulate blend on a rotary tablet press to generate tablets having the composition in the table below.

Example 8: Wet Granulation, total tablet weight = 1,222 mg

| | | | | |
|---|---|---|---|---|
| Acetaminophen (active ingredient #1) | 650 | mg | 53.2 | wt % |
| Ibuprofen (active ingredient #2) | 400 | mg | 32.7 | wt % |
| Polyvinylpyrrolidone (non-crosslinked PVP, | 110 | mg | 9 | wt % |
| povidone) | | | | |
| Sodium starch glycolate | 50 | mg | 4.1 | wt % |
| Magnesium stearate | 12 | mg | 1 | wt % |

In Example 9, amounts of the various ingredients for the batch are scaled in proportion to achieve the per-tablet amounts shown in the table below. Combine acetaminophen and ibuprofen and mix together. Pass the mixture through a sieve to sift out excessively large clumps. Combine pregelatinized starch (binder) and magnesium stearate (lubricant) and mix together. Pass the excipient mixture through a sieve to sift out excessively large clumps. Add the excipient blend to the acetaminophen/ibuprofen blend and mix together. Granulate the mixture by spraying with purified water and mixing. Pass the granulate through a sieve.

Dry the granulate on a fluid bed dryer and pass through a sieve. Add magnesium stearate (lubricant) to the dried granulate and place into a tumbler mixer for mixing. Test the dry granulate mixture for adequate flowability. Compress the granulate blend on a rotary tablet press to generate tablets having the composition in the table below.

Example 9: Wet Granulation, total tablet weight = 1,170 mg

| | | | | |
|---|---|---|---|---|
| Acetaminophen (active ingredient #1) | 650 | mg | 55.6 | wt % |
| Ibuprofen (active ingredient #2) | 400 | mg | 34.2 | wt % |
| Pregelatinized starch | 116 | mg | 9.9 | wt % |
| Magnesium stearate | 4 | mg | 0.3 | wt % |

In Example 10, amounts of the various ingredients for the batch are scaled in proportion to achieve the per-capsule amounts shown in the table below. Combine ibuprofen and acetaminophen and mix together. Add magnesium stearate (lubricant) to the powder blend and continue mixing together until thoroughly mixed. Fill capsules of size “00” with the dry powder blend.

Example 10: Capsule, total content weight = 1,061 mg

| | | | | |
|---|---|---|---|---|
| Acetaminophen (active ingredient #1) | 650 | mg | 61.3 | wt % |
| Ibuprofen (active ingredient #2) | 400 | mg | 37.7 | wt % |
| Magnesium stearate (lubricant) | 11 | mg | 1 | wt % |

### 11. CONCLUSION

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A pharmaceutical tablet consisting of:
360-440 mg of ibuprofen;
900-1,100 mg of acetaminophen;
excipient material consisting of one or more excipients that includes binder material comprising microcrystalline cellulose, pregelatinized starch, or povidone;
wherein the total amount of excipient material is less than 17 wt % of the tablet;
wherein, of the total amount of excipient material, the binder material constitutes at least 70 wt % of the excipient material.

2. The pharmaceutical tablet of claim 1, wherein the amount of binder material is less than 15 wt % of the tablet.

3. The pharmaceutical tablet of claim 1, wherein the tablet is film-coated.

4. The pharmaceutical tablet of claim 1, wherein the excipient material includes sodium starch glycolate, croscarmellose sodium, or crospovidone as a disintegrant.

5. The pharmaceutical tablet of claim 1, wherein the tablet comprises about 400 mg of ibuprofen and about 1,000 mg of acetaminophen.

6. The pharmaceutical tablet of claim 1, wherein the binder material comprises microcrystalline cellulose.

7. The pharmaceutical tablet of claim 1, wherein the binder material comprises povidone.

8. The pharmaceutical tablet of claim 1, wherein the ibuprofen and acetaminophen constitutes at least 80 wt % of the tablet.

9. The pharmaceutical tablet of claim 8, wherein the ibuprofen and acetaminophen constitutes at least 86 wt % of the tablet.

10. The pharmaceutical tablet of claim 1, wherein the total amount of binder material in the tablet is less than 11 wt % of the tablet.

11. The pharmaceutical tablet of claim 1, wherein the tablet has an elongate shape with a length of 23 mm or less and a width of 14 mm or less.

12. The pharmaceutical tablet of claim 11, wherein the tablet has a length of 21 mm or less and a width of 12 mm or less.

13. A method of treating pain in a patient, comprising:
ingesting or administering a dose consisting of a single tablet which consists of:
360-440 mg of ibuprofen;
900-1,100 mg of acetaminophen;
excipient material consisting of one or more excipients that includes binder material comprising microcrystalline cellulose, pregelatinized starch, or povidone;
wherein the total amount of excipient material is less than 17 wt % of the tablet;
wherein, of the total amount of excipient material, the binder material constitutes at least 70 wt % of the excipient material.

14. The method of claim 13, wherein the dose is a first dose, and further comprising ingesting or administering a second dose consisting of another single tablet within a time that is 3.5-6.5 hours after the first dose.

15. The method of claim 14, wherein the second dose is taken at a time that is about 4-6 hours after the first dose.

16. The method of claim 13, wherein multiple doses are taken at a frequency of every about 4-6 hours.

17. The method of claim 13, wherein the maximum adult dose amount is 3 or 4 tablets in a 24 hour period.

18. The method of claim 13, wherein the pain is acute dental pain.

19. The method of claim 18, wherein the dose is taken after a dental procedure.

20. A pharmaceutical capsule of standard size 00 or 000 and having contents consisting of:
360-440 mg of ibuprofen;
900-1,100 mg of acetaminophen;
less than 5 wt % of excipient material or lacking any excipient material.
